(19) United States
(12) Patent Application Publication
(10) Pub. No.: US 2025/0213385 A1
(43) Pub. Date: Jul. 3, 2025

(54) Excrement Collector and Nursing Machine thereof

(71) Applicant: SUZHOU ALTON ELECTRICAL & MECHANICAL INDUSTRY CO., LTD., Suzhou, Jiangsu (CN)

(72) Inventors: Weidong LU, West Chicago, IL (US); Qiang SONG, Suzhou, Jiangsu (CN); Dengke LEI, Suzhou, Jiangsu (CN); Shuhua SANG, Suzhou, Jiangsu (CN); Wei LI, Suzhou, Jiangsu (CN)

(73) Assignee: SUZHOU ALTON ELECTRICAL & MECHANICAL INDUSTRY CO., LTD., Suzhou, Jiangsu (CN)

(21) Appl. No.: 18/881,566

(22) PCT Filed: Jul. 4, 2023
(86) PCT No.: PCT/CN2023/105666

§ 371 Date: Jan. 6, 2025

(30) Foreign Application Priority Data
Jul. 5, 2022 (CN) 202210793924.6
Sep. 20, 2022 (CN) 202211145747.7
Sep. 20, 2022 (CN) 202222491928.7

(51) Int. Cl. A61F 5/451; A61F 5/44; A61M 1/00

(52) U.S. Cl. CPC A61F 5/451; A61F 5/4408; A61M 1/60; A61M 1/77

(57) ABSTRACT

An excrement collector and a nursing machine are provided. The excrement collector includes a pants assembly for wearing on a human body, and a working head that is able to be for being detachably butted together with the pants assembly. The working head includes an excretion recess for accommodating human excreta. The pants assembly includes an excretion opening for human excretion, and a connecting frame for being detachably butted together with the working head. In a state where the connecting frame of the pants assembly is butted together with the working head, the connecting frame is attached to the working head. The working head is able to be forward butted to the connecting frame of the pants assembly so as to be connected together with the pants assembly.

### CROSS-REFERENCE TO RELATED APPLICATION

The disclosure claims priority to the Chinese patent application No. 202210793924.6, filed with the Patent Office of the People's Republic of China on Jul. 5, 2022, the Chinese patent application No. 202222491928.7, filed with the Patent Office of the People's Republic of China on Sep. 20, 2022, and the Chinese patent application No. 202211145747.7, filed with the Patent Office of the People's Republic of China on Sep. 20, 2022. All contents of the above three patent applications are incorporated by reference in the disclosure.

### TECHNICAL FIELD

The disclosure relates to an excrement collector and a nursing machine thereof, in particular to connection between a working head and a pants assembly of the excrement collector.

### BACKGROUND

A nursing machine is device for sucking and clearing excreta of a human body, and is mainly used for clearing excreta of a patient to relieve the burden of a nursing worker. The nursing machine usually includes an excrement collector, a host, and a pipeline device connected between the excrement collector and the host. The excrement collector includes a working head and a pants assembly for wearing on the human body. The pipeline device usually includes a sewage suction pipeline, a water supply pipeline, a power supply cable, etc. The host usually includes a negative pressure source (namely a suction device), a sewage bucket for accommodating sewage, a clean water bucket for providing cleaning water, etc. The working head includes an excretion recess for receiving human excreta. When the excrement collector is worn on the human body for use, the human body excretes in the excretion recess, the suction device sucks the excreta received by the excrement collector through the pipeline device, and the excreta is stored in the sewage bucket. The existing pants assembly and working head are complicated in connection and inconvenient in use, and the use experience of a user needs to be improved.

### SUMMARY

One of the technical problems solved by the disclosure is to improve the convenience of connection between a pants assembly and a working head of an excrement collector.

An embodiment of the disclosure provides an excrement collector, including a pants assembly for wearing on a human body, and a working head that is able to be for being detachably butted together with the pants assembly. The working head includes an excretion recess for accommodating human excreta. The pants assembly includes an excretion opening for human excretion, and a connecting frame for being detachably butted together with the working head. In a state where the connecting frame of the pants assembly is butted together with the working head, the connecting frame is attached to the working head. The working head is able to be forward butted to the connecting frame of the pants assembly so as to be connected together with the pants assembly. With this design, the connection between the working head and the pants assembly is relatively simple, so that the use experience of a user is improved.

The excrement collector further includes a locking mechanism for locking the working head and the pants assembly. Along with the working head is forward plugged to the pants assembly, the locking mechanism locks the working head and the connecting frame together. Thus, the working head is able to be directly and quickly forward inserted and locked to the pants assembly, thereby achieving the quick connection between the working head and the pants assembly.

The disclosure further provides a nursing machine, including a host and the above excrement collector.

### BRIEF DESCRIPTION OF THE DRAWINGS

FIG. 1 is a three-dimensional view of an excrement collector from one perspective in the disclosure;

FIG. 2 is a three-dimensional view of an excrement collector from another perspective in the disclosure;

FIG. 3 is a three-dimensional view of a pants assembly in the disclosure;

FIG. 4 is a three-dimensional view in a state where a working head and a pants assembly are separated in the disclosure;

FIG. 5 is an exploded view of a pants assembly from one perspective in the disclosure;

FIG. 6 is an exploded view of a pants assembly from another perspective in the disclosure;

FIG. 7 is a three-dimensional view in a state where a working head, a connecting plug assembly, a sewage suction pipe joint, a plug, a connecting frame and a urine hood are combined together in the disclosure;

FIG. 8 is a three-dimensional view in a state where a working head is separated from a connecting plug assembly, a sewage suction pipe joint and a plug in the disclosure;

FIG. 9 is an exploded view of a working head in the disclosure;

FIG. 10 is a horizontal cross-sectional view in a state where a working head and a connecting frame are combined together in the disclosure;

FIG. 11 is a vertical cross-sectional view in a state where a working head and a connecting frame are combined together in the disclosure;

FIG. 12 is another exploded view of a working head in the disclosure;

FIG. 13 is a three-dimensional view in a state where a first housing, a second housing and a lower cover are separated in the disclosure;

FIG. 14 is a three-dimensional view in a state where a first housing and a second housing are separated in the disclosure;

FIG. 15 is a three-dimensional view of a connecting frame from one perspective in the disclosure;

FIG. 16 is a three-dimensional view of a connecting frame from another perspective in the disclosure;

FIG. 17 is an exploded view of a connecting frame from one perspective in the disclosure;

FIG. 18 is an exploded view of a connecting frame from another perspective in the disclosure; and

FIG. 19 is a three-dimensional view of a left buckle member and a right buckle member in the disclosure.

### DETAILED DESCRIPTION OF THE EMBODIMENTS

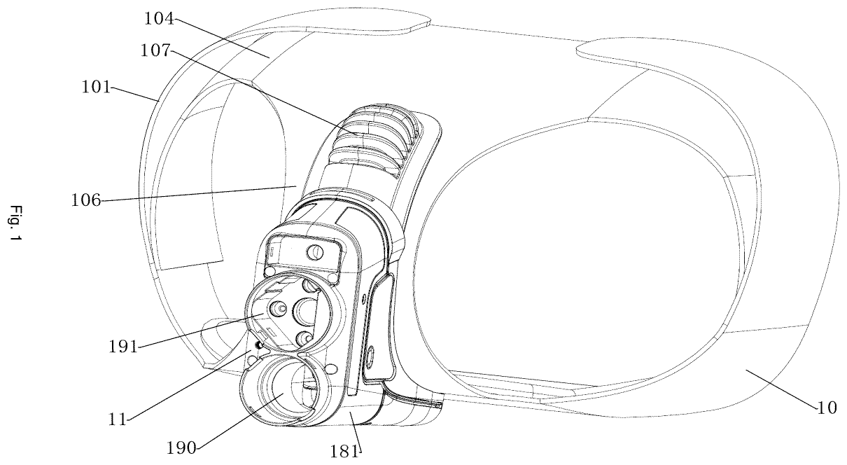

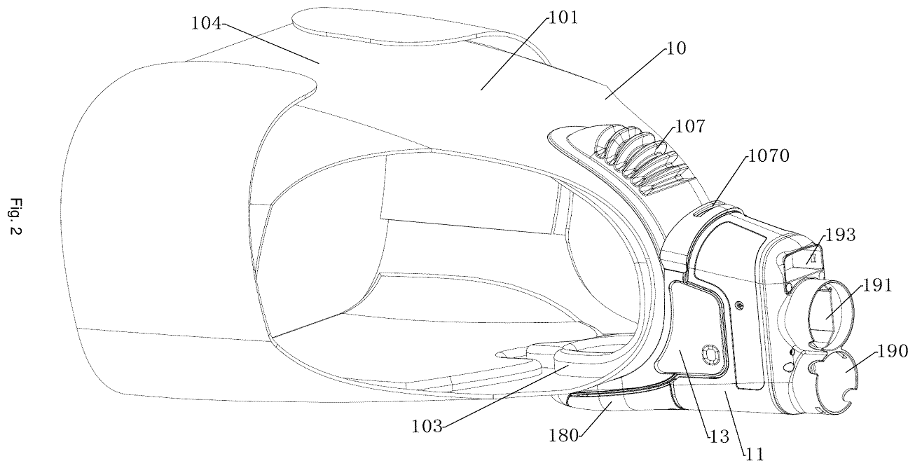

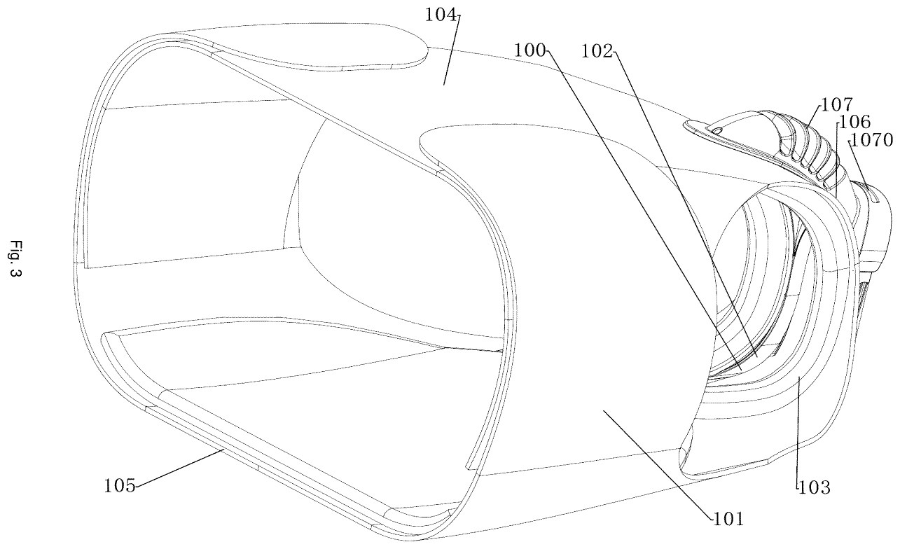

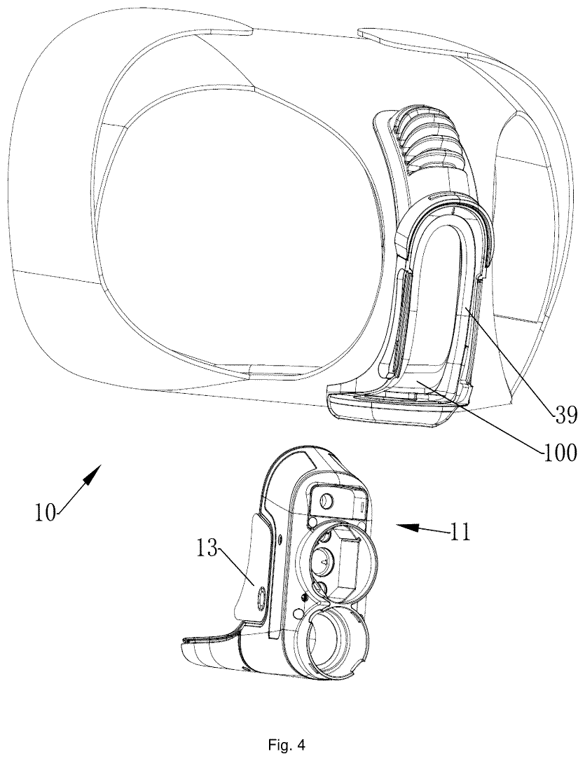

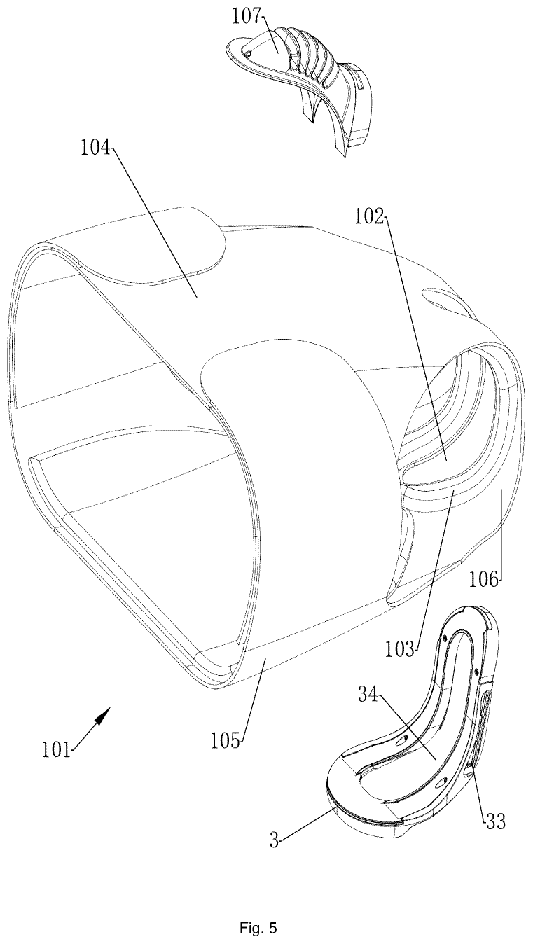

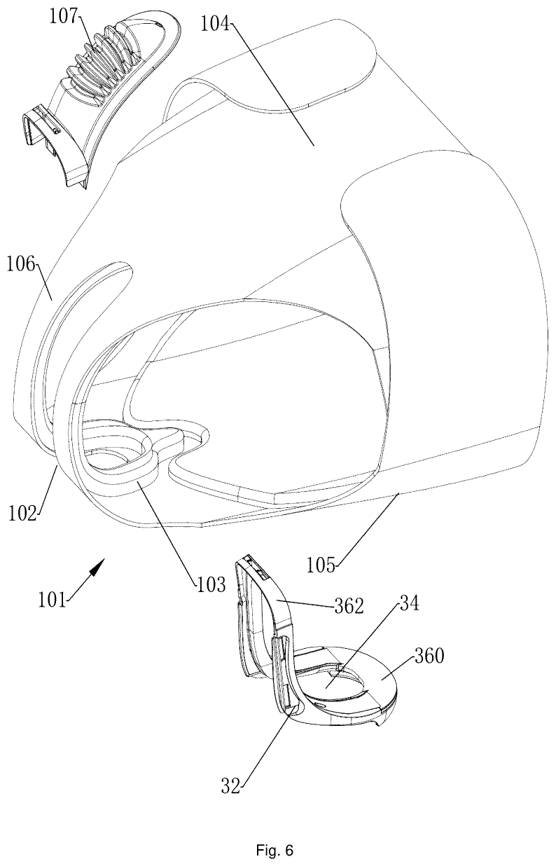

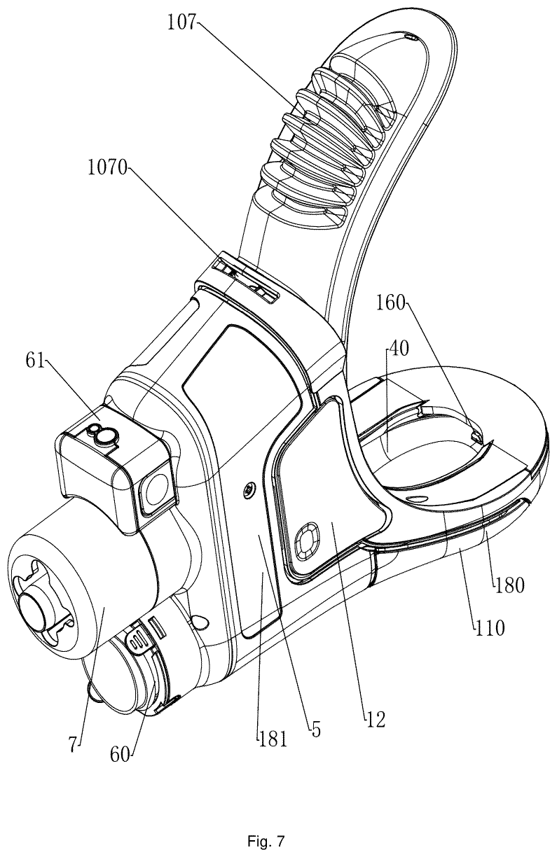

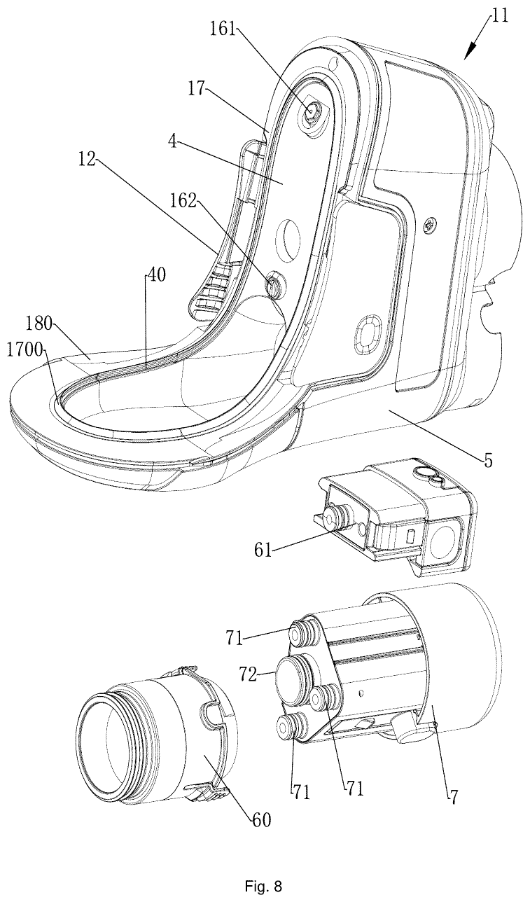

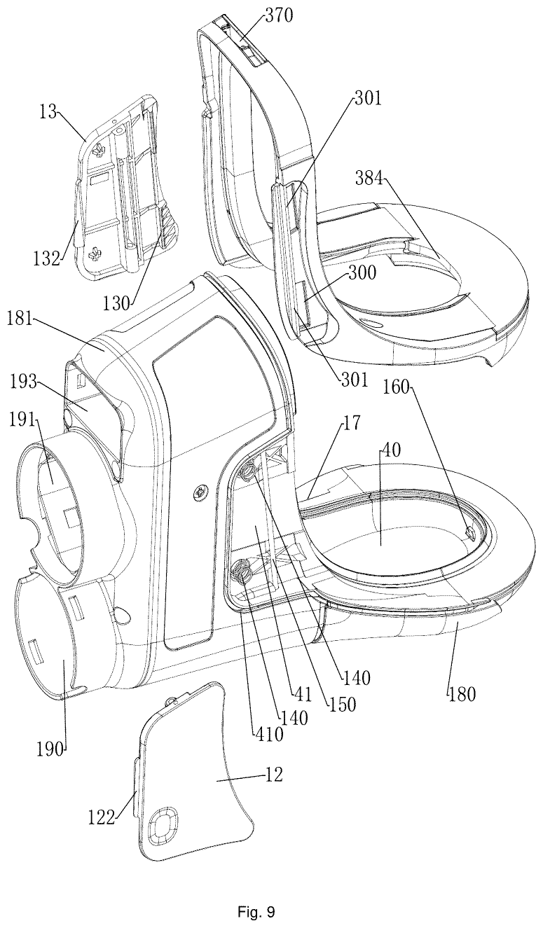

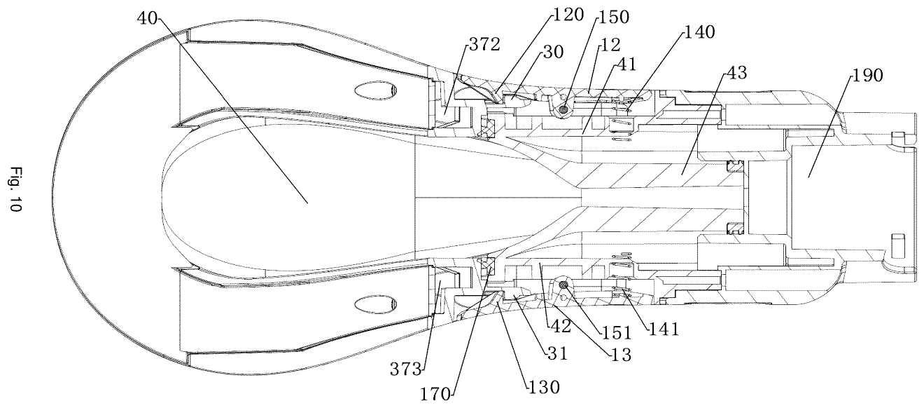

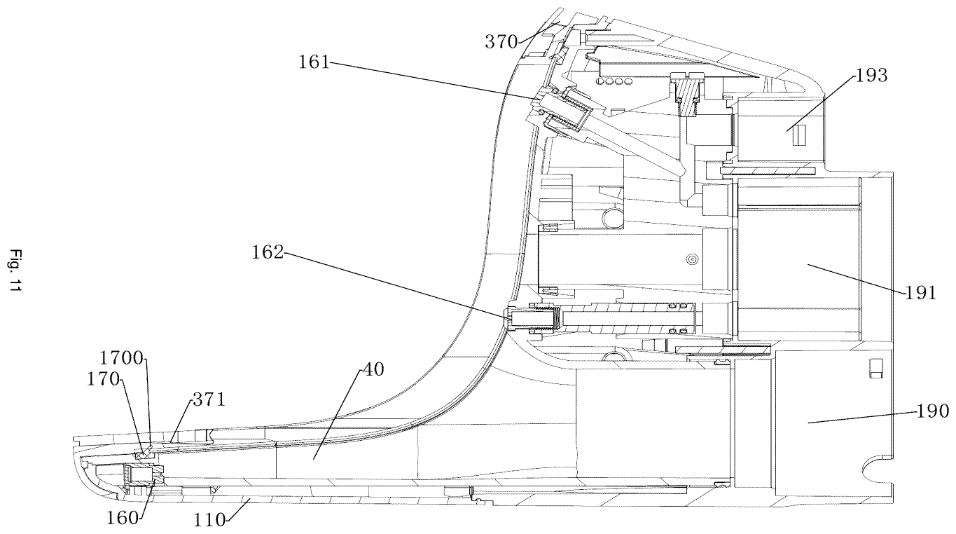

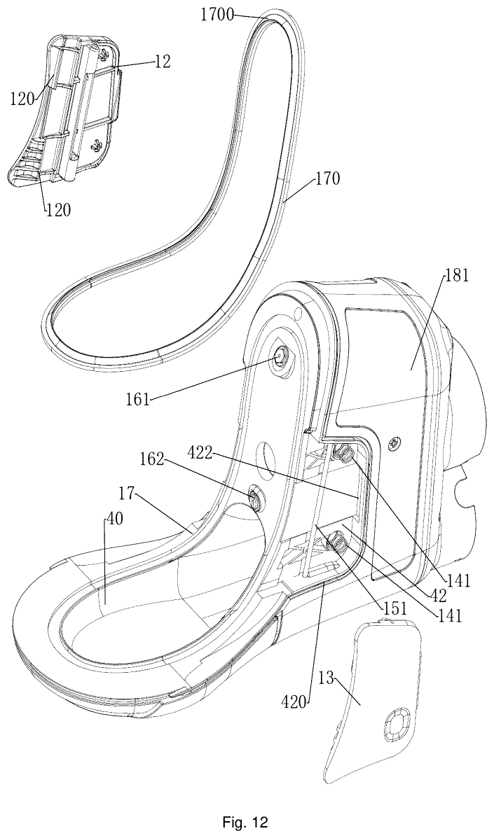

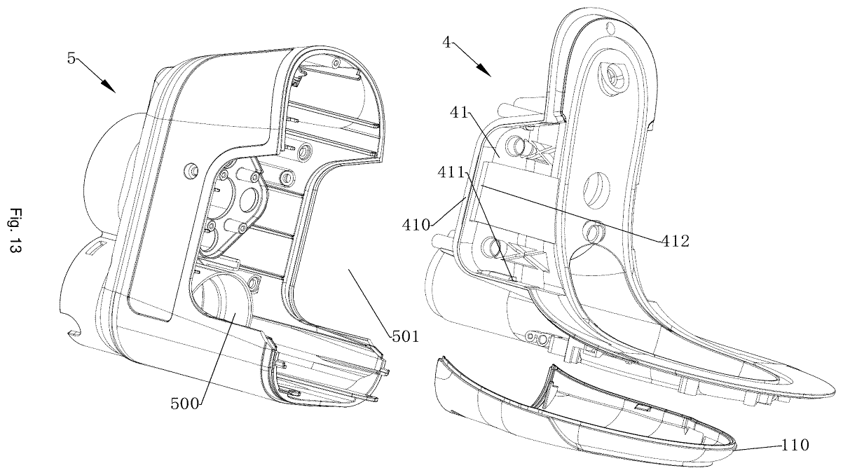

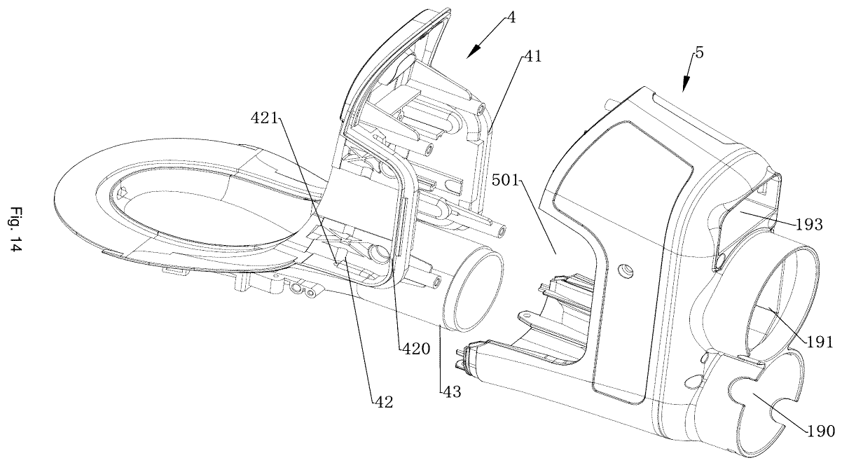

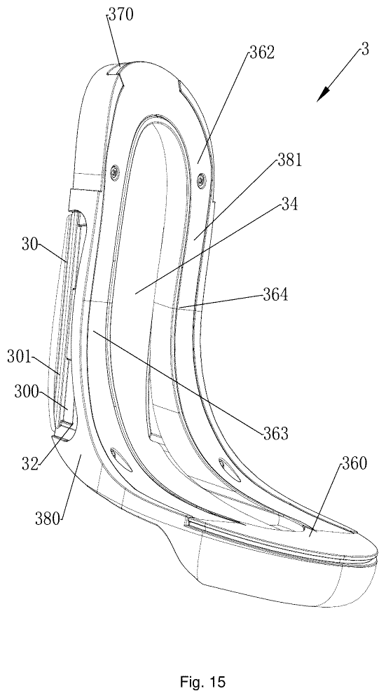

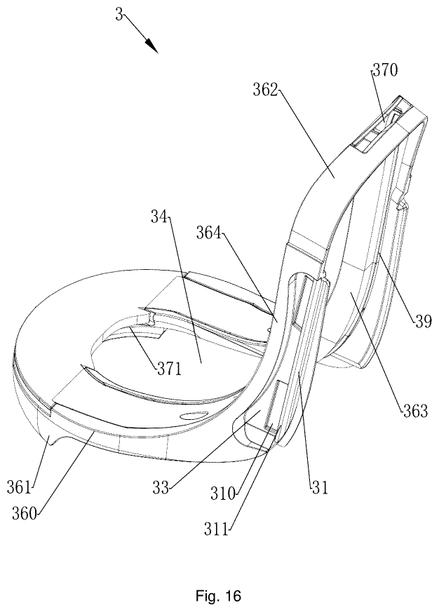

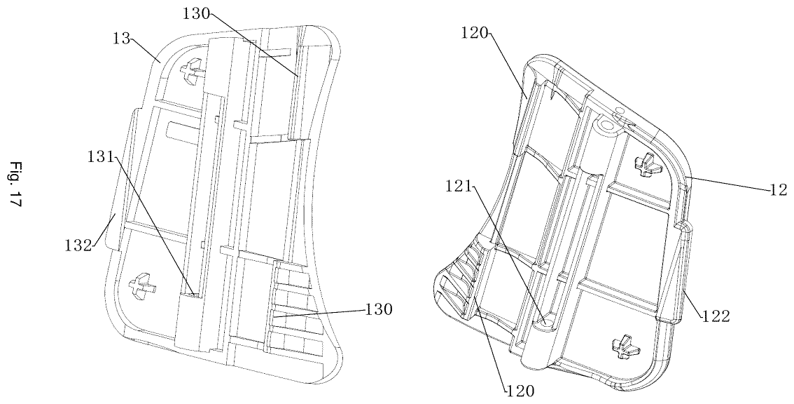

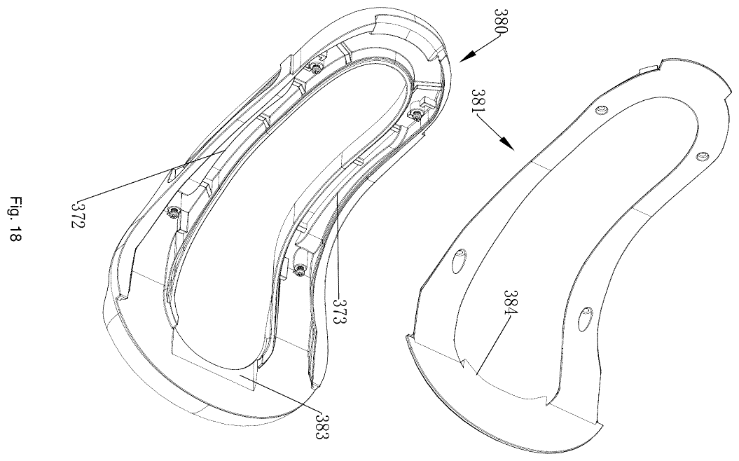

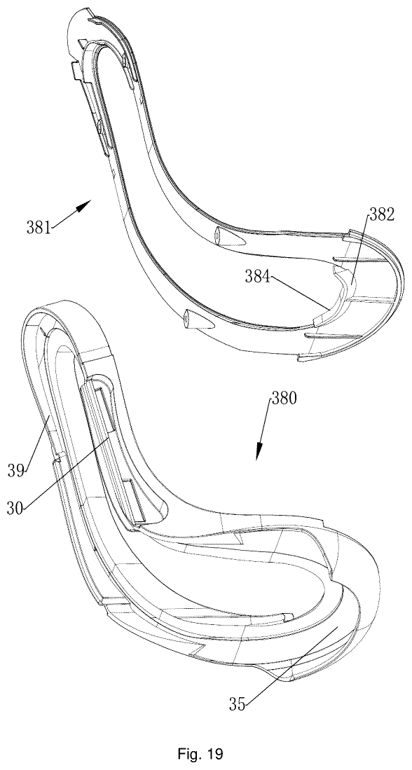

Referring to FIG. 1 to FIG. 19, an excrement collector for a nursing machine is disclosed. The excrement collector includes a pants assembly 10 for wearing on a human body, a working head 11 that is able to be for being detachably butted together with the pants assembly 10, and a locking mechanism for locking the working head 11 and the pants assembly 10. The working head 11 includes an excretion recess 40 for accommodating human excreta. The pants assembly 10 includes an excretion opening 100 for human excretion. After the pants assembly 10 is worn on the human body, the excretion part of the human body is able to be exposed to the excretion opening 100 for excretion through the excretion opening 100. The working head 11 is able to be indirectly worn on the human body by the pants assembly 10. The pants assembly 10 includes a connecting frame 3, the pants assembly 10 is able to be for being detachably butted together with the working head 11 by the connecting frame 3, and the working head 11 is able to be forward butted to the connecting frame 3 of the pants assembly 10 so as to be connected together with the pants assembly 10. The working head 11 is pluggable forward and rearward relative to the pants assembly 10, and the working head 11 is able to be forward plugged to the pants assembly 10. The working head 11 is able to be forward plugged to the pants assembly 10 by being forward plugged to the connecting frame 3 of the pants assembly 10. The working head 11 is plugged to the pants assembly 10, so that the excretion recess 40 is butted with the excretion opening 100.

The insertion and locking of the working head 11 is able to be completed simultaneously. Along with the working head 11 is forward plugged to the pants assembly 10, the locking mechanism locks the working head 11 and the connecting frame 3 together. A nursing worker is able to wear the pants assembly 10 on the human body, similar to wearing diapers on the human body, with low difficulty. Whether the pants assembly 10 is worn in place is able to be determined by direct observation through the excretion opening 100. When it is found that the pants assembly 10 is not worn in place (for example, the excretion opening 100 is deflected), the pants assembly 10 is able to be adjusted in time to prevent the pants assembly 10 from being not worn in place, thereby preventing the subsequent working head from being not worn in place, and reducing the probability of the resulting problems such as leakage of excreta. The pants assembly 10 is able to be worn on the human body in advance. When the working head 11 needs to be connected (based on the willingness of the human body for excretion), the working head 11 is able to be directly forward plugged to the pants assembly 10 very quickly and conveniently. The working head 11 and the pants assembly 10 is able to be quickly connected. The working head 11 is able to be directly inserted and fixed to the pants assembly 10. The working head 11 is able to be quickly connected more timely in response to the excretion requirement, thereby improving the use experience of a patient. Based on this design, since the working head 11 is able to be quickly and simply connected, during the routine time when excreta does not need to be received, the pants assembly 10 is able to be worn on the human body, but the working head 11 is not connected so as to enable the human body to be more relaxed and comfortable, and improve the use experience of the patient. When the excreta needs to be received, the working head 11 is quickly connected to the pants assembly 10. After the excrement collector is worn on the human body, the human body is able to excrete feces into the excretion recess 40.

The connecting frame 3 surrounds the excretion opening 100. In a state where the connecting frame 3 is butted together with the working head 11, the connecting frame 3 is attached to the working head 11. The locking mechanism includes a left buckle member 12, a left mating part 30 for snapping with the left buckle member 12, a right buckle member 13, and a right mating part 31 for snapping with the right buckle member 13. The left buckle member 12 and the right buckle member 13 belong to the working head 11 and are arranged on left and right sides of the working head 11, and the left mating part 30 and the right mating part 31 belong to the connecting frame 3 and are arranged on left and right sides of the connecting frame 3. In a state where the connecting frame 3 is butted together with the working head 11, the left buckle member 12 snaps with the left mating part 30, and the right buckle member 13 snaps with the right mating part 31, thereby locking the working head 11 and the connecting frame 3 together. During unlocking, the left buckle member 12 is able to be pressed to enable the left buckle member 12 to tilt leftward relative to the left mating part 30 so as to release the snapping fit from the left mating part 30, and the right buckle member 13 is able to be pressed to enable the right buckle member 13 to tilt rightward relative to the right mating part 31 so as to release the snapping fit from the right mating part 31. The working head 11 is pluggable with one hand relative to the pants assembly 10, and the working head 11 is able to also be unlocked with one hand. Specifically, a user is able to place one hand at the bottom of the working head 11, and fingers is able to press the left buckle member 12 and the right buckle member 13 in opposite directions to enable the left buckle member 12 and the right buckle member 13 to tilt to achieve unlocking.

The connecting frame 3 includes a left concave region 32 and a right concave region 33. The left mating part 30 is located at the left concave region 32, and the right mating part 31 is located at the right concave region 33. In a state where the connecting frame 3 is butted together with the working head 11, the left buckle member 12 snapped with the left mating part 30 is partially accommodated in the left concave region 32, and the right buckle member 13 snapped with the right mating part 31 is partially accommodated in the right concave region 33. Further, in a state where the connecting frame 3 is butted together with the working head 11, a front end of the left buckle member 12 is embedded in the left concave region 32, and a front end of the right buckle member 13 is embedded in the right concave region 33. The left buckle member 12 includes at least one left snap protrusion 120 for snapping with the left mating part 30, and the left mating part 30 includes a snap groove 300 and/or a convex rib 301. The right buckle member 13 includes at least one right snap protrusion 130 for snapping with the right mating part 31, and the right mating part 31 includes a snap groove 310 and/or a convex rib 311. The left snap protrusion 120 and the right snap protrusion 130 are formed with guide slopes. By the guide slopes, the left snap protrusion 120 and the right snap protrusion 130 more smoothly slide over the convex rib 301 and the convex rib 311 so as to be fastened with the convex rib 301 and the convex rib 311. In a state where the connecting frame 3 is butted together with the working head 11, the left snap protrusion 120 is embedded in the corresponding snap groove 300 so as to be snapped with the convex rib 301 on an edge of the corresponding snap groove 300, and the right mating part 31 is embedded in the corresponding snap groove 300 so as to be snapped with the convex rib 301 on an edge of the corresponding snap groove 300.

The left buckle member 12 and the right buckle member 13 are rotatable, although the angle of rotation is relatively small. The working head 11 further includes a left elastic resetting member 140 corresponding to the left buckle member 12 and a right elastic resetting member 141 corresponding to the right buckle member 13. In a state where the connecting frame 3 is butted together with the working head 11, the left buckle member 12 is able to be kept in a state of being snapped with the left mating part 30 under the action of the elastic force of the left elastic resetting member 140, and the right buckle member 13 is able to be kept in a state of being snapped with the right mating part 31 under the action of the elastic force of the right elastic resetting member 141. Along with the working head 11 is forward plugged to the pants assembly 10, the left buckle member 12 snaps with the left mating part 30 under the action of the elastic force of the left elastic resetting member 140, and the right buckle member 13 snaps with the right mating part 31 under the action of the elastic force of the right elastic resetting member 141. The left elastic resetting member 140 and the right elastic resetting member 141 is able to use springs. In the process of forward plugging the working head 11 to the pants assembly 10, the left buckle member 12 is resisted by the left mating part 30 and tilts leftward relative to the left mating part 30, and then is rightward snapped with the left mating part 30 under the action of the elastic force of the left elastic resetting member 140; and the right buckle member 13 is resisted by the right mating part 31 and tilts rightward relative to the right mating part 31, and then is leftward snapped with the right mating part 31 under the action of the elastic force of the right elastic resetting member 141. Thus, along with the working head 11 is inserted, the left buckle member 12 and the right buckle member 13 are basically synchronously and automatically snapped with the corresponding left mating part 30 and right mating part 31. The left buckle member 12 that tilts leftward is able to reset rightward under the action of the elastic force of the left elastic resetting member 140, and the right buckle member 13 that tilts rightward is able to reset leftward under the action of the elastic force of the right elastic resetting member 141.

The left snap protrusion 120 protrudes rightward, and the left mating part 30 protrudes leftward. In the process of forward plugging the working head 11 to the pants assembly 10, under the resisting of the left mating part 30 to the left snap protrusion 120, the left buckle member 12 tilts leftward relative to the left mating part 30, and the left snap protrusion 120 slides over the left mating part 30 and resets rightward together with the left buckle member 12 to snap with the left mating part 30. The right snap protrusion 130 protrudes leftward, and the right mating part 31 protrudes rightward. In the process of forward plugging the working head 11 to the pants assembly 10, under the resisting of the right mating part 31 to the right snap protrusion 130, the right buckle member 13 tilts rightward relative to the right mating part 31, and the right snap protrusion 130 slides over the right mating part 31 and resets leftward together with the right buckle member 13 to snap with the right mating part 31.

In summary, the locking mechanism includes at least one buckle member and at least one mating part that is able to be fastened correspondingly (in this embodiment, the mating part is divided into the left mating part 30 and the right mating part 31, and the buckle member is divided into the left buckle member 12 and the right buckle member 13), the buckle member belongs to a part of the working head 11, the mating part belongs to a part of the connecting frame 3, the buckle member is deflectable, and the buckle member is able to reset under the action of the elastic force of an elastic resetting member (such as a spring). Along with the working head 11 is forward plugged to the pants assembly 10, the buckle member snaps with the mating part under the pushing of the elastic force of the corresponding elastic resetting member. Further, along with the working head 11 is forward plugged to the pants assembly 10, the buckle member resets under the pushing of the elastic force of the corresponding elastic resetting member so as to be snapped with the mating part.

The working head 11 includes a first housing 4, a second housing 5, and a lower cover 110. The excretion recess 40 is arranged on the first housing 4. Each of the first housing 4, the second housing 5, and the lower cover 110 is one-piece component that is integrally formed. The second housing 5 is located at a rear side of the first housing 4. The lower cover 110 is mounted on a bottom side of the first housing 4 and is located at a front side of the second housing 5. The first housing 4 and the second housing 5 are connected together in a front-and-rear butting manner. The left buckle member 12 is rotatably connected to the first housing 4 or the second housing 5, and the right buckle member 13 is rotatably connected to the first housing 4 or the second housing 5. The left buckle member 12 is located at a left front side of the second housing 5, and the right buckle member 13 is located at a right front side of the second housing 5.

In this embodiment, the left buckle member 12 and the right buckle member 13 are rotatably connected to the first housing 4 respectively. The first housing 4 includes a left side plate 41 and a right side plate 42. The left side plate 41 and the right side plate 42 are located at left and right sides of the first housing 4, and the left side plate 41 and the right side plate 42 are opposite in a left-and-right spaced manner. The second housing 5 includes a left side notch 500 and a right side notch 501. The left side plate 41 is located at a right side of the left side notch 500, and the left side plate 41 blocks the left side notch 500. The right side plate 42 is located at a left side of the right side notch 501, and the right side plate 42 blocks the right side notch 501. The left buckle member 12 is located at a left side of the left side plate 41 and covers the left side plate 41, and the right buckle member 13 is located at a right side of the right side plate 42 and covers the right side plate 42. The left elastic resetting member 140 is compressed between the left buckle member 12 and the left side plate 41 to keep a leftward push against the left buckle member 12 leftward. The right elastic resetting member 141 is compressed between the right buckle member 13 and the right side plate 42 to keep a leftward push against the right buckle member 13 rightward. The space between the left side plate 41 and an edge of the left side notch 500 is sealed with glue, and the space between the right side plate 42 and an edge of the right side notch 501 is sealed with glue. The left side plate 41 is embedded in the left side notch 500, and the right side plate 42 is embedded in the right side notch 501. The left side notch 500 is located at the left front side of the second housing 5, and the right side notch 501 is located at the right front side of the second housing 5. A front end of the left side notch 500 and a front end of the right side notch 501 are respectively opened forward. As the first housing 4 is rearward mounted to the second housing 5, the left side plate 41 is rearward mounted to the left side notch 500, and the right side plate 42 is rearward mounted to the right side notch 501. The left side notch 500 and the right side notch 501 are respectively in a U shape with a front end opened forward. The left side plate 41 includes a left side blocking rib 410 protruding leftward, the left side blocking rib 410 extends along an edge of the left side plate 41, the left side blocking rib 410 fences the left buckle member 12, and the left side blocking rib 410 is spaced between an edge of the left side notch 500 and the left buckle member 12. The right side plate 42 includes a right side blocking rib 420 protruding rightward, the right side blocking rib 420 extends along an edge of the right side plate 42, the right side blocking rib 420 fences the right buckle member 13, and the right side blocking rib 420 is spaced between an edge of the right side notch 501 and the right buckle member 13. The left side blocking rib 410 and the right side blocking rib 420 are respectively U-shaped.

The left buckle member 12 is rotatably connected to the left side plate 41, and the right buckle member 13 is rotatably connected to the right side plate 42. The working head 11 includes a left side rotating shaft 150 that forms a rotating shaft of the left buckle member 12 and a right side rotating shaft 151 that forms a rotating shaft of the right buckle member 13. The left buckle member 12 is rotatably connected to the left side plate 41 by the left side rotating shaft 150, and the right buckle member 13 is rotatably connected to the right side plate 42 by the right side rotating shaft 151. The left buckle member 12 includes a first left side shaft hole 121 corresponding to the left side rotating shaft 150, and the right buckle member 13 includes a first right side shaft hole 131 corresponding to the right side rotating shaft 151. The left side rotating shaft 150 is mounted on the left side plate 41 and penetrates through the first left side shaft hole 121, and the right side rotating shaft 151 is mounted on the right side plate 42 and penetrates through the first right side shaft hole 131. The left buckle member 12 and the right buckle member 13 are rotatably connected to the first housing 4 and then the left buckle member 12, the right buckle member 13 and the first housing 4, as a whole are assembled together with the second housing 5. The left side rotating shaft 150 and the right side rotating shaft 151 extend respectively along an up-and-down direction. The left side rotating shaft 150 and the left side rotating shaft 150 is able to be made of metal materials (such as stainless steel), aluminum alloy materials, etc., so as to provide higher strength compared to the first housing 4, second housing 5 and lower cover 110 made of plastic materials.

The left buckle member 12 is in loose fit with the left side rotating shaft 150, so that the left buckle member 12 is able to rotate relative to the left side rotating shaft 150. The right buckle member 13 is in loose fit with the right side rotating shaft 151, so that the right buckle member 13 is able to rotate relative to the right side rotating shaft 151. The left side rotating shaft 150 is fixed to the left side plate 41, and the right side rotating shaft 151 is fixed to the right side plate 42. The left side plate 41 includes a plurality of second left side shaft holes 411 for mounting the left side rotating shaft 150, and the right side plate 42 includes a plurality of second right side shaft holes 421 for mounting the right side rotating shaft 151. The left side rotating shaft 150 penetrates through the second left side shaft holes 411, and the right side rotating shaft 151 penetrates through the second right side shaft holes 421. The second left side shaft holes 411 are distributed on upper and lower sides of the left buckle member 12, and the second right side shaft holes 421 are distributed on upper and lower sides of the right buckle member 13. The left side rotating shaft 150 is in interference fit with the second left side shaft hole 411 distributed on one of the upper and lower sides of the left buckle member 12 and is in loose fit with the second left side shaft hole 411 distributed on the other side of the upper and lower sides of the left buckle member 12, so that the left side rotating shaft 150 is inserted and mounted in the second left side shaft holes 411. The right side rotating shaft 151 is in interference fit with the second right side shaft hole 421 distributed on one of the upper and lower sides of the right buckle member 13 and is in loose fit with the second right side shaft hole 421 distributed on the other side of the upper and lower sides of the right buckle member 13, so that the right side rotating shaft 151 is inserted and mounted in the second right side shaft holes 421. In this embodiment, a lower end of the left side rotating shaft 150 is in interference fit with the second left side shaft hole 411 distributed on a lower side of the left buckle member 12, and during mounting, the left side rotating shaft 150 is upward inserted and fixedly mounted in the second left side shaft holes 411; and a lower end of the right side rotating shaft 151 is in interference fit with the second right side shaft hole 421 distributed on a lower side of the right buckle member 13, and during mounting, the right side rotating shaft 151 is upward inserted and fixedly mounted in the second right side shaft holes 421. The second left side shaft holes 411 are provided by the left side blocking rib 410, and the second right side shaft holes 421 are provided by the right side blocking rib 420.

The left snap protrusion 120 and the left elastic resetting member 140 are located at front and rear sides of the left side rotating shaft 150, wherein the left snap protrusion 120 is located at front of the left side rotating shaft 150, and the left elastic resetting member 140 is located behind the left side rotating shaft 150. The right snap protrusion 130 and the right elastic resetting member 141 are located at front and rear sides of the right side rotating shaft 151, wherein the right snap protrusion 130 is located at front of the right side rotating shaft 151, and the right elastic resetting member 141 is located behind the right side rotating shaft 151.

The left side plate 41 includes a left side limiting groove 412, and a part of the left buckle member 12 extends into the left side limiting groove 412. The right side plate 42 includes a right side limiting groove 422, and a part of the right buckle member 13 extends into the right side limiting groove 422. After the working head 11 is separated from the pants assembly 10, under the pushing of the left elastic resetting member 140 to the left buckle member 12, the part of the left buckle member 12 leftward abuts against a left side inner wall of the left side limiting groove 412, and under the pushing of the right elastic resetting member 141 to the right buckle member 13, the part of the right buckle member 13 rightward abuts against a right side inner wall of the right side limiting groove 422. The left side limiting groove 412 and the right side limiting groove 422 are rearward concave grooves respectively. The part of the left buckle member 12 forms a first convex part 122 protruding rearward at a rear end of the left buckle member 12, and the part of the right buckle member 13 forms a second convex part 132 protruding rearward at a rear end of the right buckle member 13. The left side limiting groove 412 is located at the left side blocking rib 410, and the right side limiting groove 422 is located at the right side blocking rib 420.

A first frame opening 34 is provided and surrounded by the connecting frame 3, and the first frame opening 34 forms at least a part of the excretion opening 100. In a state where the connecting frame 3 is butted together with the working head 11, the first frame opening 34 is butted together with the excretion recess 40 to jointly receive human excreta. The pants assembly 10 includes a pants main part 101. The connecting frame 3 is fixed to the pants main part 101. The pants main part 101 includes a crotch opening 102. The connecting frame 3 is arranged at an edge of the crotch opening 102. The first frame opening 34 forms a part of the excretion opening 100. The first frame opening 34 and the crotch opening 102 are superposed together to form the excretion opening 100. The excretion opening 100 penetrates through the connecting frame 3, and the connecting frame 3 is pasted and fixed to the pants main part 101.

The pants main part 101 includes a first flexible gasket 103 for attaching to the human body. The first flexible gasket 103 surrounds the edge of the crotch opening 102. The first flexible gasket 103 plays a sealing role between the human body and the pants main part 101, so that the leakage of excreta is not easy to occur between the human body and the pants main part 101. The pants main part 101 includes an upper wrapping sheet 104, a lower wrapping sheet 105, and a connecting part 106. The connecting part 106 is connected with the upper wrapping sheet 104 and the lower wrapping sheet 105. The crotch opening 102 is provided by the connecting part 106. The connecting frame 3 and the first flexible gasket 103 are located at two opposite sides of the connecting part 106. The upper wrapping sheet 104 and the lower wrapping sheet 105 respectively include magic tapes, so that the pants assembly 10 is able to be tightly worn on the human body. In a state where the connecting frame 3 is butted together with the working head 11, the first flexible gasket 103, the connecting part 106, the connecting frame 3, and the working head 11 are sequentially distributed from front to rear.

The connecting frame 3 includes a slot 35, the slot 35 is located at a bottom side of a front part of the connecting frame 3, the slot 35 is opened rearward, a front end of the working head 11 is able to be forward inserted into the slot 35, and the left mating part 30 and the right mating part 31 are located at a rear upper side of the slot 35. As the front end of the working head 11 is forward inserted into the slot 35, the buckle member snaps with the mating part, thereby locking the working head 11 and the connecting frame 3 together. Further, as the front end of the working head 11 is forward inserted into the slot 35, the left buckle member 12 snaps with the right mating part 31, and the right buckle member 13 snaps with the right mating part 31, thereby locking the working head 11 and the connecting frame 3 together.

The connecting frame 3 includes a first U-shaped part 360 and an arc-shaped retaining wall 361 for forming the slot 35. The arc-shaped retaining wall 361 is integrally connected to the first U-shaped part 360. The arc-shaped retaining wall 361 and the slot 35 are located at a bottom side of the first U-shaped part 360. The slot 35 is formed between the first U-shaped part 360 and the arc-shaped retaining wall 361. The front end of the working head 11 is attached to the arc-shaped retaining wall 361 after being inserted into the slot 35. The arc-shaped retaining wall 361 uses a thin wall. After the front end of the working head 11 is inserted into the slot 35, a bottom surface of the working head 11 is substantially flush with a low point of an outer surface of the arc-shaped retaining wall 361.

The connecting frame 3 includes a second U-shaped part 362, a left connecting part 363, and a right connecting part 364. The first U-shaped part 360, the left connecting part 363, the second U-shaped part 362, and the right connecting part 364 are sequentially connected to form a closed loop, thereby surrounding the first frame opening 34. The first U-shaped part 360 is located at a front lower side of the connecting frame 3, and the second U-shaped part 362 is located at a rear upper side of the connecting frame 3. The left connecting part 363 and the right connecting part 364 are located at left and right sides of the connecting frame 3, and the left connecting part 363 and the right connecting part 364 respectively extend upward from the first U-shaped part 360 to the second U-shaped part 362. The left mating part 30 is arranged at the left connecting part 363, and the right mating part 31 is arranged at the right connecting part 364. The left concave region 32 is arranged at the left connecting part 363, and the right concave region 33 is arranged at the right connecting part 364.

The pants assembly 10 includes a urine hood 107. The urine hood 107 is located above the excretion recess 40. The urine hood 107 and the excretion recess 40 are opposite vertically. An edge of a front side of the urine hood 107 is pasted on the edge of the crotch opening 102. The urine hood 107 is located between the second U-shaped part 362 and a part of the crotch opening 102. The urine hood 107 is made of a flexible silicone material. The first U-shaped part 360, the left connecting part 363, and the right connecting part 364 are pasted on the edge of the crotch opening 102. The second U-shaped part 362 is pasted on an edge of a rear end of the urine hood 107.

The connecting frame 3 includes a supplementary air inlet 370, a supplementary air outlet 371, and an airflow cavity channel. The airflow cavity channel is formed inside the connecting frame 3. The supplementary air inlet 370 and the supplementary air outlet 371 are in communication through the airflow cavity channel. The supplementary air inlet 370 forms an air inlet of the airflow cavity channel, and the supplementary air outlet 371 forms an air outlet of the airflow cavity channel. The supplementary air outlet 371 is used for supplementing air to the excretion recess 40. When the air pressure in the excretion recess 40 is negative pressure relative to the outside of the excrement collector, the air outside the excrement collector sequentially passes through the supplementary air inlet 370, the airflow cavity channel and the supplementary air outlet 371 to be supplemented into the excretion recess 40. The airflow cavity channel forms an inner cavity of the connecting frame 3. After the excrement collector is worn on the human body, the human body is able to excrete into the excretion recess 40. A negative pressure source of the host sucks the excrement collector, causing the air pressure in the excretion recess 40 to be negative pressure relative to the outside of the excrement collector, so that the excreta inside the excretion recess 40 is able to be sucked away by the negative pressure source. The air pressure in the excretion recess 40 being negative pressure relative to the outside of the excrement collector is basically caused by the suction of the negative pressure source.

The airflow cavity channel includes a left side cavity channel 372 communicating the supplementary air inlet 370 with the supplementary air outlet 371, and a right side cavity channel 373 communicating the supplementary air inlet 370 with the supplementary air outlet 371. The left side cavity channel 372 and the right side cavity channel 373 are connected to form an annular cavity channel surrounding the periphery of the first frame opening 34. When the air pressure in the excretion recess 40 is negative pressure relative to the outside of the excrement collector, part of the air sequentially passes through the supplementary air inlet 370, the left side cavity channel 372 and the supplementary air outlet 371 to be supplemented into the excretion recess 40, and another part of the air sequentially passes through the supplementary air inlet 370, the right side cavity channel 373 and the supplementary air outlet 371 to be supplemented into the excretion recess 40.

The working head 11 includes a front side nozzle 160. The front side nozzle 160 is located at a front side of the excretion recess 40. The front side nozzle 160 is used for rearward spraying water into the excretion recess 40 so as to flush excreta rearward. The front side nozzle 160 is adjacent to the supplementary air outlet 371. The front side nozzle 160 is located at a lower side of the supplementary air outlet 371. The supplementary air inlet 370 is formed on a top side of a rear upper end of the connecting frame 3. In a side lying state of the working head 11, the lowest point of the supplementary air inlet 370 is higher than the lowest point of the supplementary air outlet 371 to prevent the water entering the airflow cavity channel from the supplementary air outlet 371 from leaking from the supplementary air inlet 370 in the side lying state of the working head 11. The supplementary air outlet 371 is arranged at the first U-shaped part 360, and the supplementary air inlet 370 is arranged at the second U-shaped part 362. The working head 11 includes a first rear side nozzle 161 for spraying water and a second rear side nozzle 162 for spraying water. The urine hood 107 includes a through hole 1070. The through hole 1070 corresponds to the supplementary air inlet 370. A part of the urine hood 107 wraps the second U-shaped part 362. The through hole 1070 is provided by the part of the urine hood 107. The part of the urine hood 107 is able to be prevented from blocking the supplementary air inlet 370 by the through hole 1070. The airflow cavity channel is in communication with the excretion recess 40 by the supplementary air outlet 371, and the airflow cavity channel is in communication with the outside of the excrement collector by the supplementary air inlet 370 and the through hole 1070. When the air pressure in the excretion recess 40 is negative pressure relative to the outside of the excrement collector, the air outside the excrement collector sequentially passes through the through hole 1070, the supplementary air inlet 370, the airflow cavity channel and the supplementary air outlet 371 to be supplemented into the excretion recess 40. The front side nozzle 160, the first rear side nozzle 161, and the second rear side nozzle 162 are respectively mounted on the first housing 4.

The connecting frame 3 is hollow. The connecting frame 3 includes a first frame body and a second frame body. The airflow cavity channel is formed between the first frame body and the second frame body. The first frame body and the second frame body are combined together to form the connecting frame 3. The first frame body forms a closed loop, and the second frame body forms a closed loop. Further, the connecting frame includes a main frame body 380 that forms the first frame body and a front cover plate 381 that forms the second frame body (that is, in this embodiment, the first frame body is the main frame body 380, and the second frame body is the front cover plate 381), and the front cover plate 381 is closed on the main frame body 380, thereby forming the connecting frame 3. The supplementary air inlet 370, the supplementary air outlet 371, and the airflow cavity channel are respectively formed between the front cover plate 381 and the main frame body 380. The front cover plate 381 includes a first guide surface 382, and the main frame body 380 includes a second guide surface 383. A gap between the first guide surface 382 and the second guide surface 383 forms the supplementary air outlet 371. The first guide surface 382 is inclined rearward and downward to guide the airflow of the supplementary air outlet 371 to flow rearward and downward, and the second guide surface 383 is inclined rearward and downward to guide the airflow of the supplementary air outlet 371 to flow rearward and downward.

The second frame body includes a baffle plate 384 corresponding to the supplementary air outlet 371. The baffle plate 384 rearward extends into the first frame opening 34. The first guide surface 382 forms at least a part of a bottom surface of the baffle plate 384. The second guide surface 383 is located at a lower side of the baffle plate 384. The inclination angle of the first guide surface 382 is greater than that of the second guide surface 383. The main frame body 380 is a one-piece component that is integrally formed. The left mating part 30, the right mating part 31, the arc-shaped retaining wall 361, the left concave region 32, and the right concave region 33 respectively belong to a part of the main frame body 380.

The connecting frame 3 has a first attaching side 39, and the working head 11 has a second attaching side 17. The excretion recess 40 is concave inward the working head 11 from the second attaching side 17. In a state where the connecting frame 3 is butted together with the working head 11, the first attaching side 39 is attached to the second attaching side 17. The left mating part 30 and the right mating part 31 are located at left and right sides of the first attaching side 39, and the left buckle member 12 and the right buckle member 13 are located at left and right sides of the second attaching side 17. One of the first attaching side 39 and the second attaching side 17 includes a second flexible gasket 170. The pants assembly 10 includes a third attaching side 108 for attaching to the human body, and the excretion opening 100 penetrates from the third attaching side 108 to the first attaching side 39. The second flexible gasket 170 includes a convex tilted edge 1700 for attaching to the other one of the first attaching side 39 and the second attaching side 17. The tilted edge 1700 forms a loop corresponding to the first frame opening 34. The tilted edge 1700 is inclined towards the excretion recess 40 and is in a closed state. The second flexible gasket 170 is able to be made of a flexible silicone material. The second flexible gasket 170 plays a sealing role between the connecting frame 3 and the working head 11 to prevent excreta from leaking between the connecting frame 3 and the working head 11. The first flexible gasket 103 is arranged on the third attaching side 108.

The working head 11 includes a base part 180 and a rear seat part 181. The base part 180 extends forward from the rear seat part 181. The rear seat part 181 extends upward and is higher than the base part 180. The excretion recess 40 is concavely arranged at the base part 180. A top side of the base part 180 and a front side of the rear seat part 181 form the second attaching side 17 for attaching to the connecting frame 3. In a state where the connecting frame 3 is butted together with the working head 11, the base part 180 is located at a lower side of the connecting frame 3, and the rear seat part 181 is located at a rear side of the connecting frame 3. Along with the working head 11 is forward plugged to the pants assembly 10, the front side of the rear seat part 181 is forward attached to the connecting frame 3. A part of the first housing 4 forms a top wall of the base part 180, and another part of the first housing 4 forms a front wall of the rear seat part 181. The second housing 5 at least forms a top wall, a bottom wall, a left side wall, a right side wall and a rear wall of the rear seat part 181. The front side nozzle 160 is arranged at the base part 180, and the first rear side nozzle 161 and the second rear side nozzle 162 are arranged at the rear seat part 181. The first housing 4 includes a drainage pipe connected to the excretion recess 40. The lower cover 110 belongs to a part of the base part 180.

The disclosure further provides a nursing machine, including a host (not shown), the above excrement collector, a sewage suction pipe device (not shown) for connecting the host and the working head 11, and a pipeline device (not shown) for connecting the host and the working head 11. The working head 11 includes a drainage interface 190 and an adapting interface 191. The drainage interface 190 is parallel to the adapting interface 191 and is located at a lower side of the adapting interface 191. The sewage suction pipe device includes a sewage suction pipe joint 60 and a sewage suction hose (not shown) connected between the sewage suction pipe joint 60 and the host. The sewage suction pipe joint 60 is pluggable relative to the working head 11. The sewage suction pipe joint 60 is able to be forward inserted and butted to the drainage interface 190 so as to be in fluid communication with the excretion recess 40. The pipeline device includes a connecting plug assembly 7 for butting to the working head 11, at least one water supply pipe (not shown) that is able to be used for supplying water to the working head 11, an air delivery pipe (not shown) that is able to be used for delivering warm air to the working head 11, and at least one power supply cable (not shown). The connecting plug assembly 7 is pluggable relative to the working head 11. The pipeline device is connected to the adapting interface 191 by the connecting plug assembly 7. The connecting plug assembly 7 is able to be forward inserted and butted to the adapting interface 191. The host provides electric energy to the working head 11, and the working head 11 wirelessly obtains power. The working head 11 includes a first coil module (not shown). The connecting plug assembly 7 includes a second coil module (not shown) corresponding to the first coil module, at least one water supply port 71 correspondingly connected to the at least one water supply pipe, and an air supply port 72 correspondingly connected to the air delivery pipe. The at least one power supply cable is electrically connected between the host and the second coil module. The working head 11 receives the wireless power supply of the second coil module by the first coil module. In a state where the connecting plug assembly 7 is butted together with the adapting interface 191, the first coil module and the second coil module are opposite in a spaced manner in a direction perpendicular to a front-and-rear direction. In a state where the connecting plug assembly 7 is butted together with the adapting interface 191, the first coil module is coupled with the second coil module to receive the wireless power supply of the second coil module. The front side nozzle 160, the first rear side nozzle 161, and the second rear side nozzle 162 are respectively in communication with one water supply port 71 corresponding to the fluid.

The host supplies water to the working head 11 through the water supply pipe so as to clean the human excretion part and the excretion recess 40. The host is able to provide warm air to the working head 11 through the air delivery pipe so as to dry the human body, etc. The drainage interface 190 and the adapting interface 191 are arranged at the rear seat part 181. From another perspective, the drainage interface 190 and the adapting interface 191 are arranged on the second housing 5. The first housing 4 includes a drainage pipe 43. The drainage pipe 43 is located at a rear side of the excretion recess 40 and is in communication with the fluid in the excretion recess 40. The drainage pipe 43 extends rearward. The drainage pipe 43 is connected between the excretion recess 40 and the drainage interface 190. The drainage interface 190 is located at a rear side of the drainage pipe 43. The drainage pipe 43 and the drainage interface 190 are connected together in a front-and-rear butting manner. A plug 61 is used for being plugged to a standby interface 193.

The nursing machine includes wheels, and the wheels are located at the bottom of the host so as to move the host. The negative pressure source includes a motor and an impeller. The impeller rotates under the drive of the motor to generate suction airflow so as to suck the excreta received by the excretion recess 40. Under the suction of the negative pressure source, the air pressure in the excretion recess 40 is negative pressure relative to the outside of the excrement collector, and the sewage in the excretion recess 40 is sucked into the host by the sewage suction pipe device.

1. An excrement collector, comprising a pants assembly for wearing on a human body, and a working head that is able to be for being detachably butted together with the pants assembly, wherein the working head comprises an excretion recess for accommodating human excreta; the pants assembly comprises an excretion opening for human excretion, and a connecting frame for being detachably butted together with the working head;
the excrement collector further comprises a locking mechanism for locking the working head and the pants assembly, wherein the locking mechanism comprises a left buckle member, a left mating part for snapping with the left buckle member, a right buckle member, and a right mating part for snapping with the right buckle member; the left buckle member and the right buckle member belong to the working head and are arranged on left and right sides of the working head, and the left mating part and the right mating part belong to the connecting frame and are arranged on left and right sides of the connecting frame; and in a state where the connecting frame is butted together with the working head, the left buckle member snaps with the left mating part, and the right buckle member snaps with the right mating part, thereby locking the working head and the connecting frame together.

2. The excrement collector according to claim 1, wherein the working head is able to be indirectly worn on the human body by the pants assembly, the connecting frame is located at a crotch of the pants assembly, and the connecting frame surrounds the excretion opening; the working head comprises a base part, a rear seat part, a front side nozzle, and a first rear side nozzle; the base part extends forward from the rear seat part, the rear seat part extends upward and is higher than the base part, the excretion recess is concavely arranged at the base part, the front side nozzle is arranged at the base part and is located at a front side of the excretion recess, and the first rear side nozzle are arranged at the rear seat part; and in a state where the connecting frame is butted together with the working head, the base part is located at a lower side of the connecting frame, and the rear seat part is located at a rear side of the connecting frame;
the working head comprises a first housing, a second housing and a lower cover, the excretion recess is arranged on the first housing, the second housing is located at a rear side of the first housing, the left buckle member is rotatably connected to the first housing or the second housing, the right buckle member is rotatably connected to the first housing or the second housing, the left buckle member is located at a left front side of the second housing, and the right buckle member is located at a right front side of the second housing;
each of the first housing, the second housing, and the lower cover is one-piece component that is integrally formed, the lower cover is mounted on a bottom side of the first housing and is located at a front side of the second housing;
a part of the first housing forms a top wall of the base part, and another part of the first housing forms a front wall of the rear seat part, the lower cover belongs to a part of the base part;
the left buckle member is located at a left front side of the base part, and the right buckle member is located at a right front side of the base part.

3. The excrement collector according to claim 1, wherein a first frame opening is provided and surrounded by the connecting frame, and the first frame opening forms at least a part of the excretion opening; in a state where the connecting frame is butted together with the working head, the first frame opening is butted together with the excretion recess; and after the excrement collector is worn on the human body, the human body is able to excrete feces into the excretion recess;
in a state where the connecting frame of the pants assembly is butted together with the working head, the connecting frame is attached to the working head; and the working head is able to be forward butted to the connecting frame of the pants assembly so as to be connected together with the pants assembly, the excrement collector further comprises a locking mechanism for locking the working head and the pants assembly, wherein the working head is pluggable forward and rearward relative to the pants assembly; and along with the working head is forward plugged to the pants assembly, the locking mechanism locks the working head and the connecting frame of the pants assembly together.

4. The excrement collector according to claim 1, wherein the pants assembly further comprises a pants main part, the connecting frame is fixed to the pants main part, the pants main part comprises a crotch opening, a first frame opening is provided and surrounded by the connecting frame, the first frame opening forms a part of the excretion opening, and the first frame opening and the crotch opening are superposed together to form the excretion opening;
the pants main part further comprises a first flexible gasket for attaching to the human body, and the pants main part comprises an upper wrapping sheet, a lower wrapping sheet, and a connecting part; the connecting part is connected with the upper wrapping sheet and the lower wrapping sheet, the crotch opening is provided by the connecting part, and the connecting frame and the first flexible gasket are located at two opposite sides of the connecting part; and in a state where the connecting frame is butted together with the working head, the first flexible gasket, the connecting part, the connecting frame, and the working head are sequentially distributed from front to rear.

5-7. (canceled)

8. The excrement collector according to claim 1, wherein the connecting frame comprises a left concave region and a right concave region; the left mating part is located at the left concave region, and the right mating part is located at the right concave region; and in a state where the connecting frame is butted together with the working head, the left buckle member snaps with the left mating part is partially accommodated in the left concave region, and the right buckle member snaps with the right mating part is partially accommodated in the right concave region;
in a state where the connecting frame is butted together with the working head, a front end of the left buckle member is embedded in the left concave region, and a front end of the right buckle member is embedded in the right concave region.

9-10. (canceled)

11. The excrement collector according to claim 1, wherein the left buckle member and the right buckle member are rotatable; the working head comprises a left elastic resetting member corresponding to the left buckle member and a right elastic resetting member corresponding to the right buckle member; and in a state where the connecting frame is butted together with the working head, the left buckle member is kept in a state of being snapped with the left mating part under the action of the elastic force of the left elastic resetting member, and the right buckle member is kept in a state of being snapped with the right mating part under the action of the elastic force of the right elastic resetting member.

12. The excrement collector according to claim 11, wherein along with the working head is forward plugged to the pants assembly, the left buckle member snaps with the left mating part under the action of the elastic force of the left elastic resetting member, and the right buckle member snaps with the right mating part under the action of the elastic force of the right elastic resetting member;
in the process of forward plugging the working head to the pants assembly, the left buckle member is resisted by the left mating part and tilts leftward relative to the left mating part, and then is rightward snapped with the left mating part under the action of the elastic force of the left elastic resetting member; and the right buckle member is resisted by the right mating part and tilts rightward relative to the right mating part, and then is leftward snapped with the right mating part under the action of the elastic force of the right elastic resetting member.

13. (canceled)

14. The excrement collector according to claim 12, wherein the left buckle member comprises at least one left snap protrusion for snapping with the left mating part, the left snap protrusion protrudes rightward, and the left mating part protrudes leftward; in the process of forward plugging the working head to the pants assembly, under the resisting of the left mating part to the left snap protrusion, the left buckle member tilts leftward relative to the left mating part, and the left snap protrusion slides over the left mating part and resets rightward together with the left buckle member to snap with the left mating part; the right buckle member comprises at least one right snap protrusion for snapping with the right mating part, the right snap protrusion protrudes leftward, and the right mating part protrudes rightward; and in the process of forward plugging the working head to the pants assembly, under the resisting of the right mating part to the right snap protrusion, the right buckle member tilts rightward relative to the right mating part, and the right snap protrusion slides over the right mating part and resets leftward together with the right buckle member to snap with the right mating part.

15. (canceled)

16. The excrement collector according to claim 1, wherein the working head comprises a first housing and a second housing, the excretion recess is arranged on the first housing, the second housing is located at a rear side of the first housing, the left buckle member is rotatably connected to the first housing or the second housing, the right buckle member is rotatably connected to the first housing or the second housing, the left buckle member is located at a left front side of the second housing, and the right buckle member is located at a right front side of the second housing;
the working head comprises a lower cover, a drainage interface, and an adapting interface, the drainage interface and the adapting interface are arranged on the second housing, each of the first housing, the second housing, and the lower cover is one-piece component that is integrally formed, the lower cover is mounted on a bottom side of the first housing and is located at a front side of the second housing, and the first housing and the second housing are connected together in a front-and-rear butting manner; the first housing comprises a drainage pipe, the drainage pipe is located at a rear side of the excretion recess and is in fluid communication with the excretion recess, and the drainage pipe extends rearward; and the second housing comprises the drainage interface and the adapting interface, the drainage interface is parallel to the adapting interface and is located at a lower side of the adapting interface, the drainage pipe is connected between the excretion recess and the drainage interface, the drainage interface is located at a rear side of the drainage pipe, and the drainage pipe and the drainage interface are connected together in a front-and-rear butting manner.

17. The excrement collector according to claim 1, wherein the working head comprises a first housing and a second housing, the excretion recess is arranged on the first housing, the second housing is located at a rear side of the first housing, the left buckle member is rotatably connected to the first housing or the second housing, the right buckle member is rotatably connected to the first housing or the second housing, the left buckle member and the right buckle member are rotatably connected to the first housing respectively; the working head comprises a left elastic resetting member and a right elastic resetting member; the first housing comprises a left side plate and a right side plate; the left side plate and the right side plate are located at left and right sides of the first housing, and the left side plate and the right side plate are opposite in a left-and-right spaced manner; the second housing comprises a left side notch and a right side notch; the left side plate is located at a right side of the left side notch, and the left side plate blocks the left side notch; the right side plate is located at a left side of the right side notch, and the right side plate blocks the right side notch; the left buckle member is located at a left side of the left side plate and covers the left side plate, and the right buckle member is located at a right side of the right side plate and covers the right side plate; the left elastic resetting member is compressed between the left buckle member and the left side plate to keep a leftward push against the left buckle member; and the right elastic resetting member is compressed between the right buckle member and the right side plate to keep a leftward push against the right buckle member rightward.

18. The excrement collector according to claim 17, wherein the second housing is located at a rear side of the first housing, the left buckle member is rotatably connected to the first housing or the second housing, the right buckle member is rotatably connected to the first housing or the second housing; each of the first housing and the second housing is one-piece component that is integrally formed; the left side notch is located at the left front side of the second housing, and the right side notch is located at the right front side of the second housing; a front end of the left side notch and a front end of the right side notch are respectively opened forward; and along with the first housing is rearward mounted to the second housing, the left side plate is rearward mounted to the left side notch, and the right side plate is rearward mounted to the right side notch;
the left side plate comprises a left side blocking rib protruding leftward, the left side blocking rib extends along an edge of the left side plate, the left side blocking rib fences the left buckle member, and the left side blocking rib is spaced between an edge of the left side notch and the left buckle member; and the right side plate comprises a right side blocking rib protruding rightward, the right side blocking rib extends along an edge of the right side plate, the right side blocking rib fences the right buckle member, and the right side blocking rib is spaced between an edge of the right side notch and the right buckle member.

19. (canceled)

20. The excrement collector according to claim 17, wherein the left buckle member is rotatably connected to the left side plate, and the right buckle member is rotatably connected to the right side plate;
the working head further comprises a left side rotating shaft that forms a rotating shaft of the left buckle member and a right side rotating shaft that forms a rotating shaft of the right buckle member; the left buckle member is rotatably connected to the left side plate by the left side rotating shaft, and the right buckle member is rotatably connected to the right side plate by the right side rotating shaft; the left buckle member comprises a first left side shaft hole corresponding to the left side rotating shaft, and the right buckle member comprises a first right side shaft hole corresponding to the right side rotating shaft; and the left side rotating shaft is mounted on the left side plate and penetrates through the first left side shaft hole.

21. (canceled)

22. The excrement collector according to claim 20, wherein the right side rotating shaft is mounted on the right side plate and penetrates through the first right side shaft hole, the left buckle member and the right buckle member are rotatably connected to the first housing and then the left buckle member, the right buckle member and the first housing, as a whole, are assembled together with the second housing; the left side rotating shaft and the right side rotating shaft extend respectively along an up-and-down direction; the left buckle member is in loose fit with the left side rotating shaft, and the right buckle member is in loose fit with the right side rotating shaft; the left side rotating shaft is fixed to the left side plate, and the right side rotating shaft is fixed to the right side plate; the left side plate comprises a plurality of second left side shaft holes for mounting the left side rotating shaft, and the right side plate comprises a plurality of second right side shaft holes for mounting the right side rotating shaft; the left side rotating shaft penetrates through the second left side shaft holes, and the right side rotating shaft penetrates through the second right side shaft holes; the second left side shaft holes are distributed on upper and lower sides of the left buckle member, and the second right side shaft holes are distributed on upper and lower sides of the right buckle member; and the left side rotating shaft is in interference fit with the second left side shaft hole distributed on one of the upper and lower sides of the left buckle member and is in loose fit with the second left side shaft hole distributed on the other side of the upper and lower sides of the left buckle member, and the right side rotating shaft is in interference fit with the second right side shaft hole distributed on one of the upper and lower sides of the right buckle member and is in loose fit with the second right side shaft hole distributed on the other side of the upper and lower sides of the right buckle member;
the left side plate comprises a left side blocking rib protruding leftward, and the left side blocking rib fences the left buckle member; the right side plate comprises a right side blocking rib protruding rightward, and the right side blocking rib fences the right buckle member; and the second left side shaft holes are provided by the left side blocking rib, and the second right side shaft holes are provided by the right side blocking rib.

23. (canceled)

24. The excrement collector according to claim 17, wherein the working head further comprises a left side rotating shaft that forms a rotating shaft of the left buckle member and a right side rotating shaft that forms a rotating shaft of the right buckle member; the left side rotating shaft and the right side rotating shaft extend respectively along an up-and-down direction; the left side rotating shaft is mounted on the left side plate, and the right side rotating shaft is mounted on the right side plate; the left buckle member comprises a left snap protrusion, and the right buckle member comprises a right snap protrusion; the left snap protrusion and the right snap protrusion is able to be respectively snapped with the connecting frame; the left snap protrusion is located at front of the left side rotating shaft, and the left elastic resetting member is located behind the left side rotating shaft; and the right snap protrusion is located at front of the right side rotating shaft, and the right elastic resetting member is located behind the right side rotating shaft.

25. The excrement collector according to claim 17, wherein the left side plate comprises a left side limiting groove, and a part of the left buckle member extends into the left side limiting groove; the right side plate comprises a right side limiting groove, and a part of the right buckle member extends into the right side limiting groove; and after the working head is separated from the pants assembly, under the pushing of the left elastic resetting member to the left buckle member, the part of the left buckle member leftward abuts against a left side inner wall of the left side limiting groove, and under the pushing of the right elastic resetting member to the right buckle member, the part of the right buckle member rightward abuts against a right side inner wall of the right side limiting groove;
the part of the left buckle member forms a first convex part protruding rearward at a rear end of the left buckle member, and the part of the right buckle member forms a second convex part protruding rearward at a rear end of the right buckle member; the left side plate comprises a left side blocking rib protruding leftward, and the left side blocking rib fences the left buckle member; the right side plate comprises a right side blocking rib protruding rightward, and the right side blocking rib fences the right buckle member; and the left side limiting groove is located at the left side blocking rib, and the right side limiting groove is located at the right side blocking rib.

26. (canceled)

27. The excrement collector according to claim 1, wherein the connecting frame comprises a slot, the slot is located at a bottom side of a front part of the connecting frame, a front end of the working head is able to be forward inserted into the slot, and the left mating part and the right mating part are located at a rear upper side of the slot;
the slot is located at the bottom of the pants assembly; and along with the front end of the working head is forward inserted into the slot, the left buckle member snaps with the left mating part, and the right buckle member snaps with the right mating part, thereby locking the working head and the connecting frame together;
the connecting frame comprises a first U-shaped part and an arc-shaped retaining wall for forming the slot, the arc-shaped retaining wall and the slot are located at a bottom side of the first U-shaped part, the slot is formed between the first U-shaped part and the arc-shaped retaining wall, and the front end of the working head is attached to the arc-shaped retaining wall after being inserted into the slot.

28-29. (canceled)

30. The excrement collector according to claim 1, wherein the connecting frame comprises a first U-shaped part, a second U-shaped part, a left connecting part, and a right connecting part; the first U-shaped part, the left connecting part, the second U-shaped part, and the right connecting part are sequentially connected to form a closed loop; the first U-shaped part is located at a front lower side of the connecting frame, and the second U-shaped part is located at a rear upper side of the connecting frame; the left connecting part and the right connecting part are located at left and right sides of the excretion opening, and the left connecting part and the right connecting part respectively extend upward from the first U-shaped part to the second U-shaped part; and the left mating part is arranged at the left connecting part, and the right mating part is arranged at the right connecting part.

31. The excrement collector according to claim 30, wherein the first U-shaped part is located at a bottom side of the pants assembly, the left connecting part, and the right connecting part and the second U-shaped part are located at a rear side of the pants assembly; the working head comprises a base part and a rear seat part; the base part extends forward from the rear seat part, the rear seat part extends upward and is higher than the base part, and the excretion recess is concavely arranged at the base part; in a state where the connecting frame is butted together with the working head, the first U-shaped part is located at an upper side of the base part and is attached to the base part, and the left connecting part and the right connecting part are located at a front side of the rear seat part and are attached to the rear seat part; and the left buckle member and the right buckle member are located at left and right sides of the rear seat part;
the left buckle member comprises at least one left snap protrusion for snapping with the left mating part, and the left mating part comprises a snap groove and/or a convex rib; and the right buckle member comprises at least one right snap protrusion for snapping with the right mating part, and the right mating part comprises a snap groove and/or a convex rib.

32. The excrement collector according to claim 30, wherein the connecting frame comprises a supplementary air inlet, a supplementary air outlet, and an airflow cavity channel; the supplementary air inlet and the supplementary air outlet are in communication through the airflow cavity channel; the supplementary air outlet is used for supplementing air to the excretion recess; and the supplementary air outlet is arranged at the first U-shaped part, and the supplementary air inlet is arranged at the second U-shaped part;
the connecting frame is hollow, the airflow cavity channel is formed inside the connecting frame, and when the air pressure in the excretion recess is negative pressure relative to the outside of the excrement collector, an air outside the excrement collector sequentially passes through the supplementary air inlet, the airflow cavity channel and the supplementary air outlet to be supplemented into the excretion recess;
the working head comprises a front side nozzle, the front side nozzle is located at a front side of the excretion recess, the front side nozzle is used for rearward spraying water into the excretion recess, the front side nozzle is adjacent to the supplementary air outlet, the front side nozzle is located at a lower side of the supplementary air outlet, and the supplementary air inlet is formed on a top side of a rear upper end of the connecting frame.

33-42. (canceled)

43. A nursing machine, comprising the excrement collector, and the excrement collector comprises a pants assembly for wearing on a human body, and a working head that is able to be for being detachably butted together with the pants assembly, wherein the working head comprises an excretion recess for accommodating human excreta; the pants assembly comprises an excretion opening for human excretion, and a connecting frame for being detachably butted together with the working head;
the excrement collector further comprises a locking mechanism for locking the working head and the pants assembly, wherein the locking mechanism comprises a left buckle member, a left mating part for snapping with the left buckle member, a right buckle member, and a right mating part for snapping with the right buckle member; the left buckle member and the right buckle member belong to the working head and are arranged on left and right sides of the working head, and the left mating part and the right mating part belong to the connecting frame and are arranged on left and right sides of the connecting frame; and in a state where the connecting frame is butted together with the working head, the left buckle member snaps with the left mating part, and the right buckle member snaps with the right mating part, thereby locking the working head and the connecting frame together.

44-45. (canceled)
